(19) United States
(12) Patent Application Publication
(10) Pub. No.: US 2022/0215932 A1
(43) Pub. Date: Jul. 7, 2022

(54) SERVER FOR PROVIDING PSYCHOLOGICAL STABILITY SERVICE, USER DEVICE, AND METHOD OF ANALYZING MULTIMODAL USER EXPERIENCE DATA FOR THE SAME

(71) Applicant: ELECTRONICS AND TELECOMMUNICATIONS RESEARCH INSTITUTE, Daejeon (KR)

(72) Inventors: Kyoung Ju NOH, Daegu (KR); Jeong Muk LIM, Daejeon (KR); Chi Yoon JEONG, Daejeon (KR); Hyun Tae JEONG, Daejeon (KR); Ga Gue KIM, Daejeon (KR); Ji Youn LIM, Daejeon (KR); Seung Eun CHUNG, Daejeon (KR)

(21) Appl. No.: 17/536,953

(22) Filed: Nov. 29, 2021

(30) Foreign Application Priority Data
Jan. 5, 2021 (KR) 10-2021-0000911

(51) Int. Cl. G16H 20/70; G10L 25/63

(52) U.S. Cl. CPC G16H 20/70; G10L 25/63

(57) ABSTRACT

Provided is a method of analyzing multimodal user experience data for providing a psychological stability service, the method including: receiving multimodal user experience data collected on the basis of a user device that is predetermined; analyzing an association between the collected multimodal user experience data to identify whether a user is in a stressful situation; when the stressful situation is identified, analyzing a frequency pattern of the stressful situation of the user; recognizing stress context information about the stressful situation occurring to the user on the basis of the frequency pattern of the stressful situation; and providing the user device with a preemptive psychological stability service on the basis of the stress context information corresponding to the stressful situation.

### CROSS-REFERENCE TO RELATED APPLICATION

This application claims priority to and the benefit of Korean Patent Application No. 10-2021-0000911, filed on Jan. 5, 2021, the disclosure of which is incorporated herein by reference in its entirety.

### BACKGROUND

### 1. Field of the Invention

The present invention relates to a server for providing a psychological stability service, a user device, and a method of analyzing multimodal user experience data for the same.

### 2. Discussion of Related Art

With various life patterns and personalization, psychological stability support services are provided in various fields, such as for daily life, accidents, and employment.

Such a psychological stability support service is provided by measuring stress of each individual, conducting counseling based on the measured stress index, and providing a psychological stability support service that may relieve stress based on the counselling.

Recently, attempts have been made to provide psychological stability support services using various types of user devices, but there are still technical limitations in providing users with services at an appropriate time by utilizing and analyzing measurement data.

### SUMMARY OF THE INVENTION

The present invention is directed to providing a server for providing a psychological stability service, a user device, and a method of analyzing multimodal user experience data for the same that may collect multimodal user experience data based on a device worn or carried by a user, analyze an association between the collected experience data, recognize stress context information for a stressful situation that occurs frequently, and provide a preemptive psychological stabilization service matching the stress context information.

The technical objectives of the present invention are not limited to the above, and other objectives may become apparent to those of ordinary skill in the art based on the following description.

According to the first aspect of the present invention, there is provided a method of analyzing multimodal user experience data for providing a psychological stability service, the method including: receiving multimodal user experience data collected on the basis of a user device that is predetermined; analyzing an association between the collected multimodal user experience data to identify whether a user is in a stressful situation; when the stressful situation is identified, analyzing a frequency pattern of the stressful situation of the user; recognizing stress context information about the stressful situation occurring to the user on the basis of the frequency pattern of the stressful situation; and providing the user device with a preemptive psychological stability service on the basis of the stress context information corresponding to the stressful situation.

According to the second aspect of the present invention, there is provided a server for providing a psychological stability service by analyzing multimodal user experience data, the server including: a communication module configured to receive multimodal user experience data collected from a user device, which is predetermined, and provide the user device with a preemptive psychological stability service; a memory in which a program for providing the user device with the preemptive psychological stability service on the basis of the multimodal user experience data is stored; and a processor configured to execute the program stored in the memory, wherein the processor executes the program to: analyze an association between the collected multimodal user experience data to identify whether a user is in a stressful situation; when the stressful situation is identified, analyze a frequency pattern of the stressful situation of the user; recognize stress context information about a stressful situation occurring to the user on the basis of the frequency pattern of the stressful situation; and provide the user device with a preemptive psychological stability service on the basis of the stress context information corresponding to the stressful situation.

According to the third aspect of the present invention, there is provided a user device for performing a psychological stability service on the basis of multimodal user experience data, the user device including: a sensor unit configured to receive multimodal user experience data including one or more of: surrounding context information of a user including information related to at least one among a time, a place, a temperature, and a conversation partner; physiological signal information of the user; behavior information of the user; speech signal information of the user; and speech text information of the user; a communication module configured to transmit the multimodal user experience data to a psychological stability service providing server, and receive a preemptive psychological stability service provided from the psychological stability service providing server; a memory in which an application for providing the preemptive psychological stability service is stored; and a processor configured to execute the program stored in the memory to: analyze an association between the collected multimodal user experience data to identify whether the user is in a stressful situation; and when the stressful situation is identified, receive a preemptive psychological stability service based on stress context information corresponding to the stressful situation and execute the preemptive psychological stability service, wherein the stress context information for the stressful situation occurring to the user is recognized on the basis of a frequency pattern of the stressful situation of the user.

In addition, according to another aspect of the present invention, there is provided a computer readable recording medium on which a computer program that executes other methods and systems for implementing the present invention and the method described above is recorded.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a diagram for describing a system for providing a psychological stability service according to an embodiment of the present invention;

FIG. 2 is a block diagram for describing a user device that performs a psychological stability service;

FIG. 3 is a block diagram for describing a server for providing a psychological stability service;

FIG. 4 is a diagram for describing an example of recognizing a stressful situation based on multimodal user experience data;

FIG. 5 is a diagram for describing an example of recognizing a stressful situation based on surrounding context information, physiological signal information, and behavior information;

FIG. 6 is a diagram for describing an example of recognizing stress context information in an embodiment of the present invention; and

FIG. 7 is a flowchart showing a method of analyzing multimodal user experience data according to an embodiment of the present invention.

### DETAILED DESCRIPTION OF EXEMPLARY EMBODIMENTS

Hereinafter, the advantages and features of the present invention and ways of achieving them will become readily apparent with reference to descriptions of the following detailed embodiments in conjunction with the accompanying drawings.

However, the present invention is not limited to such embodiments and may be embodied in various forms. The embodiments to be described later are provided only to complete the disclosure of the present invention and assist those of ordinary skill in the art in fully understanding the scope of the present invention, and the scope of the present invention is defined only by the appended claims.

Terms used herein are used for aiding in the description and understanding of the embodiments and are not intended to limit the scope and spirit of the present invention. It should be understood that the singular forms “a” and “an” also include the plural forms unless the context clearly dictates otherwise. The terms “comprises,” “comprising,” “includes,” and/or “including,” when used herein, specify the presence of stated features, integers, steps, operations, elements, components and/or groups thereof and do not preclude the presence or addition of one or more other features, integers, steps, operations, elements, components, and/or groups thereof. In connection with assigning reference numerals to elements in the drawings, the same reference numerals are used for designating the same elements throughout the specification, and the term “and/or” includes any one or combinations of the associated listed items. It should be understood that, although the terms “first,” “second,” etc. may be used herein to describe various elements, these elements are not limited by these terms. These terms are only used for distinguishing one element from another. For example, a first element could be termed a second element without departing from the scope of the present invention.

Unless otherwise defined, all terms (including technical and scientific terms) used herein have the same meaning as commonly understood by one of ordinary skill in the art to which this invention belongs. It should be further understood that terms, such as those defined in commonly used dictionaries, should not be interpreted in an idealized or overly formal sense unless expressly so defined herein.

The present invention relates to a server 100 for providing a psychological stability service, a user device 200, and a method of analyzing multimodal user experience data for the same.

Recently, there is an increased use of various life-logging services and products for storing and analyzing daily experience data of users to provide users with useful services. For example, a lifelog application installed in a smartphone automatically recognizes user's location movement, activity information, mood information, etc., or receives such information input by the user based on a specific time unit and analyzes a lifestyle of the user based on the received information. The analyzed lifestyle information may be provided to the user through a specific user interface (UI) to a user device or may be linked with a function of recommending specific content.

As another embodiment of the lifelog service, wearable devices are being commercialized that operate an application for sensing exercise information and bio-signals, such as pulses, of a user through a smart wristband worn by the user to manage the amount of daily exercise of the user or for detecting abnormal signals on the user's health to link to an emergency call service.

As the user experience data-based devices and services have increased utilization, there is an increasing demand for personalized affective computing services that may improve the emotional quality of life of users in daily life.

A representative affective computing service includes a psychological stability service based on experience data of a user. The psychological stability service refers to a service for inducing a user to emotional stability in a stressful situation in which negative emotions, such as tension and anger, occur to a user. Examples of the psychological stability service include an application that periodically provides specific content to a user to induce emotional stability or an application that recommends specific content to a user according to statistics of an emotional label input by the user into a lifelogging application.

Representative technologies for emotion recognition of a user may be classified according to the type of experience data used. For example, the technologies are divided into an emotion recognition technology based on facial expression of a user, a speech-based emotion recognition technology based on speeches of a user, a physiological-based emotion recognition technology, a multimodal emotion recognition technology using various pieces of experience data of a user which are fused, etc. A change in emotion and user experience data has characteristics of individual differences between users, subjectivity of emotions, and diverse changes according to situations, and thus it is often difficult to maintain a constant and high emotion recognition rate in all situations.

As a general example of a change in emotion and speech signal, a negative emotional state, such as an angry situation, may cause the speech of the user to become louder or higher in tone and increase the heart rate, body temperature, and muscle tension compared to a usual state of the user.

Alternatively, in the case of a negative emotional state, such as depression, in general, the speech of the user has characteristics of an utterance speed being slowed, a pause time being lengthened, a pitch variation being reduced, and an amplitude being lowered.

In addition, the physiological signal has a close association with specific behavior of a user, such as sitting, walking, standing, running, and sleeping, and the ambient temperature. For example, compared to a static state, such as a sitting or standing situation, the heart rate and respiration rate of the user in a dynamic state, such as a walking or running situation, increase. In addition, the pulse rate of the user in a state of emotional excitement may be observed to be higher than usual but may also be high in a situation such as vigorous exercise or high external temperature.

In addition, as an example of the association between the temperature and the physiological response, high temperatures cause the skin blood vessels of a person to expand, resulting in an increase in skin temperature, which leads to a body reaction for releasing body heat. That is, the heart allows the circulating blood volume on the surface of the skin to increase, causing the pulse to become faster and the cardiac output to increase, resulting in a faster heartbeat.

With regard to analyzing emotion data of a user based on experience data of the user as described above, it may be efficient to use the association between pieces of experience data in various types and situations for accurate emotion recognition.

With regard to providing a psychological stability service, it is very important to identify an efficient point in time at which the psychological stability service starts operation. When the psychological stability service is provided at an inaccurate point in time, it may cause discomfort to the user, and the effect may be halved.

Assuming that a psychological stabilization service starts at a point in time at which a negative emotion of a user in a stressful situation is recognized based on experience data, an incompleteness of the actual emotion recognition result and a delay of the recognition time point may increase a chance that a psychological stabilization service is not efficiently performed at a time when the service is needed in practice.

In order to solve such a problem, an embodiment of the present invention may, in order to automatically recognize information about stress context information (Point Of Concerned Context: POCC) for a stressful situation requiring operation of a psychological stability service from accumulated multimodal user experience data and update the recognized information about the POCC, analyze the multimodal user experience data, and provide a preemptive psychological stabilization service based on the analysis.

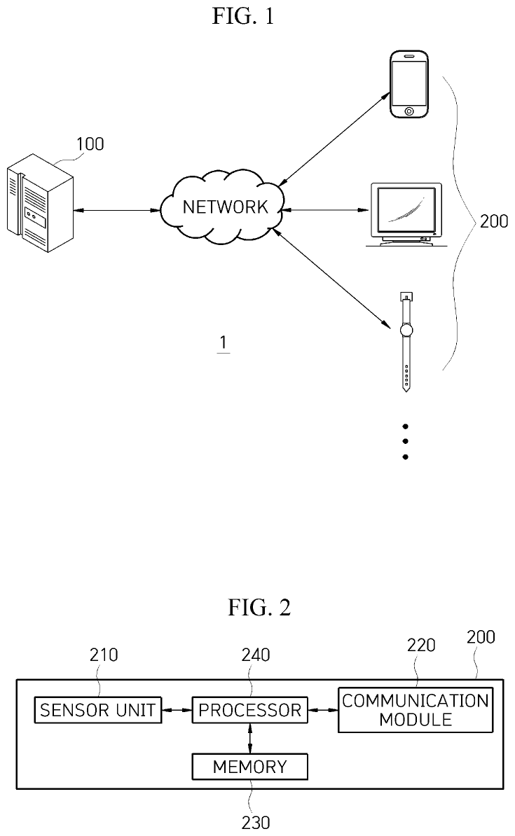

Hereinafter, a server 100 for providing a psychological stability service (hereinafter referred to as a server) according to an embodiment of the present invention will be described with reference to FIGS. 1 to 6.

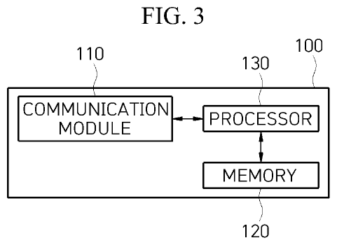

FIG. 1 is a diagram for describing a system 1 for providing a psychological stability service according to an embodiment of the present invention. FIG. 2 is a block diagram for describing a user device 200 that performs a psychological stability service. FIG. 3 is a block diagram for describing a server 100 for providing a psychological stability service.

The system 1 for providing a psychological stability service by analyzing multimodal user experience data according to the embodiment of the present invention includes the user device 200 and the server 100 which are predetermined.

In this case, each component constituting the system 1 for providing a psychological stability service shown in FIG. 1 may be connected via a network. The network refers to a connection structure in which information exchange is performed between nodes such as terminals and servers, and examples of the network include a 3rd Generation Partnership Project (3GPP) network, a Long Term Evolution (LTE) network, a World Interoperability for Microwave Access (WiMAX) network, the Internet, a Local Area Network (LAN), a Wireless LAN (WLAN), a Wide Area Network (WAN), a Personal Area Network (PAN), a Bluetooth network, a satellite broadcasting network, an analog broadcasting network, a Digital Multimedia Broadcasting (DMB) network, and the like, but the network according to the present invention is not limited thereto.

Referring to FIG. 2, the user device 200 includes a sensor unit 210, a communication module 220, a memory 230, and a processor 240.

The sensor unit 210 collects multimodal user experience data. The multimodal user experience data may be collected through the user device 200 such as a wearable device or personal portable device of the user. For example, the multimodal user experience data may be sensed and collected by one user device 200 or may be sensed and collected by the first user device and then collected by the second user device. Another embodiment may be implemented such that one part of the multimodal user experience data is sensed and collected by the first user device, and another part of the multimodal user experience data is sensed and collected by the second user device and then integrated and collected into a certain user device and delivered to the server 100.

As described above, in implementing the present invention, the collection location of the multimodal user experience data may be variously changed according to the type of the user device 200 used or the environment configuration setting of the user device 200, and in the present invention, the collection location according to the type of the multimodal user experience data to be collected and the user device 200 are not particularly limited.

Here, the multimodal user experience data includes surrounding context information SC, physiological signal information P, user behavior information B, and speech signal information SS extracted from a spoken speech of a user, and speech text information ST.

The surrounding context information SC may include information representing a situation around the user, such as a time (context time: CT), a place (context place: CP), a temperature, and conversation partner information. As an example, the sensor unit 210 may collect, as time information included in the surrounding context information, information about a specific context in a daily life of a user, such as absolute time information for a standard time, “AM” and “PM,” in the form of processed time information.

The physiological signal information P of the user may include data such as a heart rate HR, a heart rate variation HRV calculated through heart rate information, a galvanic skin response (GSR), photoplethysmogram (PPG), etc. The sensor unit 210 collects physiological signal information of the user through various physiological signal measuring sensors provided in the user device 200. According to an embodiment of the present invention, a negative emotional change, such as a stressful situation of a user, is recognized on the basis of the association between characteristics extracted based on collected physiological signal information P and multimodal user experience data, such as behavior information B of the user, which will be described below.

The behavior information B of the user may include user motion information, such as sitting and running, and high-level contextual behavior information, such as eating, moving, and sleeping. To this end, the sensor unit 210 may collect a motion sensor signal through a location information collection sensor such as an accelerometer, a geomagnetic sensor, or a gyroscope.

In addition, the sensor unit 210 may identify a speech signal of the user and an utterance sentence of the user from a sound signal collected through a built-in microphone and collect the speech signal and the utterance sentence as speech signal information SS. Based on the speech signal information SS, speech characteristics in the frequency domain, such as Mel-Frequency Cepstrum Coefficients (MFCC) and characteristics, such as the amplitude, speaking rate, emphasis, pitch variability, utterance duration, pause time, etc. of a speech in the time domain may be extracted to recognize a change in the user's emotion and, based on the speech signal information SS, speech text information ST, which is spoken text of the user, may be extracted.

The communication module 220 transmits and receives data to and from the server 100, and an application for providing a preemptive psychological stability service is installed and stored in the memory 230 in advance.

The processor 240 executes the application stored in the memory 230 to transmit multimodal user experience data collected through the communication module 220 and receive and execute a preemptive psychological stabilization service provided from the server 100.

The server 100 analyzes the association between the collected multimodal user experience data to identify whether a user is in a stressful situation, and when it is identified that the user is in a stressful situation, recognizes stress context information corresponding to the stressful situation through an analysis section having multi-dimensional time information, and when a stressful situation matches the stress context information, provides a preemptive psychological stability service to the user device 200.

Unlike the present embodiment, the processor 240 may directly analyze the association between collected multimodal user experience data by itself to identify whether the user is in a stressful situation. That is, the processor 240 may recognize a negative emotion, such as a stressful situation, of the user by linking physiological signal information, speech signal information, and speech text information with surrounding context information and behavior information. Examples of emotion attributes recognizable by the processor 240 may include specific emotion labels, such as sadness, joy, and anger, and various sentiment labels including user's biorhythms and emotions distinguished by a certain unit, such as valence and arousal, etc.

When it is identified that the user is in a stressful situation, the processor 240 may receive a preemptive psychological stabilization service based on context information corresponding to the stressful situation from the server 100 and execute the preemptive psychological stabilization service. In this case, the server 100 may recognize the stress context information for the stressful situation occurring to the user and provide the stress context information to the user device 200.

Referring to FIG. 3, the server 100 for providing a psychological stability service by analyzing multimodal user experience data includes a communication module 110, a memory 120, and a processor 130.

The communication module 110 is an element for an interface with a predetermined user device 200 and receives multimodal user data collected from the user device 200 and provides the user device 200 with a preemptive psychological stability service.

In the memory 120, a program for providing a preemptive psychological stability service based on multimodal user experience data is stored, and the processor 130 executes the program stored in the memory 120.

The processor 130 analyzes the association between the collected multimodal user experience data to identify whether the user is in a stressful situation and, when it is identified that the user is in a stressful situation, analyzes a frequency pattern of the stressful situation of the user, recognizes stress context information for a stressful situation occurring to the user on the basis of the frequency pattern of the stressful situation, and provides the user device 200 with a preemptive psychological stabilization service on the basis of the stress context information corresponding to the stressful situation.

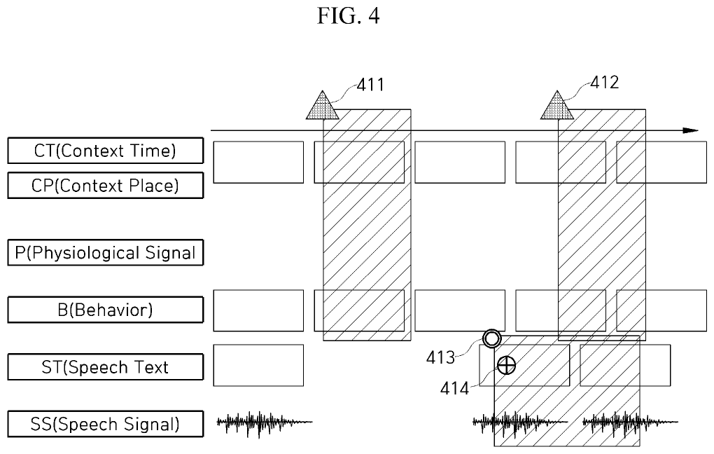

FIG. 4 is a diagram for describing an example of recognizing a stressful situation based on multimodal user experience data.

The processor 130 may recognize stressful situations 411 and 412 that are negative emotion change on the basis of surrounding context information, physiological signal information, and behavior information of a user.

In addition, the processor 130 may recognize stressful situations 413 and 414 on the basis of at least one of speech signal information and speech text information of a user. For example, when the stressful situation 413 based on the speech signal information is recognized, speech text information uttered by the user in a presentation section of the stressful situation may also be extracted.

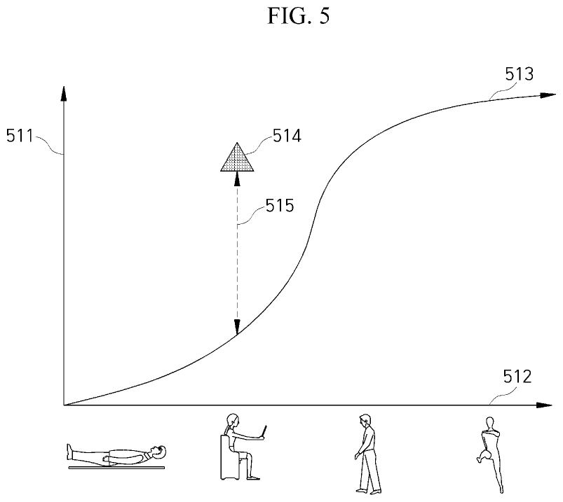

FIG. 5 is a diagram for describing an example of recognizing a stressful situation based on surrounding context information, physiological signal information, and behavior information.

In one embodiment, the processor 130 cumulatively collects physiological signal information and behavior information among the multimodal user experience data from the user device 200 and, through analysis of an association to the information, calculates an association index 513 for a change in physiological signal information 511 of the user corresponding to an activity amount 512 of each behavior, such as sleeping, sitting, walking, running, etc. of the user. When the physiological signal information 511 of the user at a specific point in time 514 according to the surrounding situation information has a variance greater than or equal to a preset threshold 515 from the association index 513, compared to the behavior information 512 of the user at the point in time 514, the specific point in time may be identified as a stressful situation 514 of the user.

In this case, as a method of analyzing an association that may be used in implementing the present invention, a method of calculating the distance between two points or a direction-based calculation method, such as cosine similarity, may be used.

Meanwhile, it should be understood that the method of analyzing an association between multimodal user experience data or the method of recognizing a stressful situation based on the analyzed association information in the present invention may be applied to various embodiments in addition to the above-described embodiment.

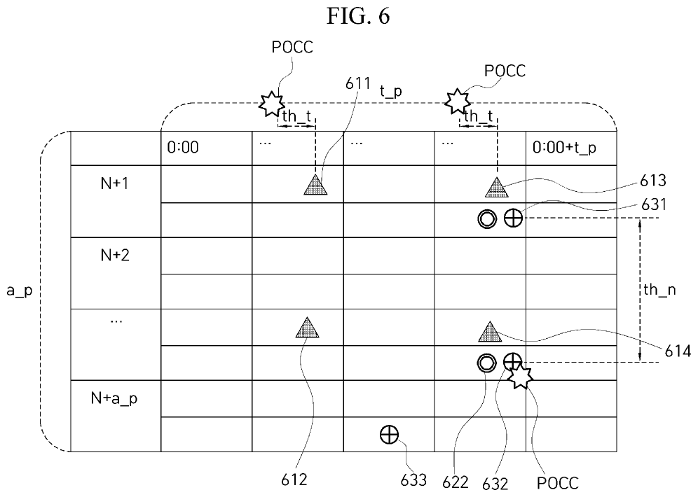

FIG. 6 is a diagram for describing an example of recognizing stress context information in an embodiment of the present invention.

As described above, the processor 130 may identify whether stressful situations 611 to 614 have occurred based on surrounding context information, physiological signal information, and behavior information of the user, and identify whether stressful situations 621, 622, 631, 632, and 633 have occurred based on speech signal information or speech text information.

In addition, the processor 130, in response to a stressful situation identified as having occurred, may analyze a frequency pattern of the stressful situation and, to this end, may analyze the frequency pattern of the stressful situation composed of multidimensional time information including a unit time period (time period: t_p) and a unit accumulation period (accumulation period: a_p).

In an embodiment of the present invention, information about recognizing a negative emotion that is a stressful situation may be managed based on multidimensional time information of a unit time period t_p and a unit accumulation period a_p. Here, a specific time within the unit time period t_p is associated with time information in surrounding context information at a time of collecting multimodal user experience data.

The processor 130 recognizes stress context information POCC for a stressful situation that occurs to a user based on the frequency pattern of the stressful situation managed based on the multidimensional time information.

To this end, the processor 130 may recognize, as the stress context information POCC, at least one of the surrounding situation information obtained before a predetermined threshold time th t from a point in time at which the stressful situation 611 to 614 is identified as having occurred and the speech text information 631 to 633 commonly uttered by the user at a level greater than or equal to a preset threshold value th_n.

In this case, the processor 130 may set a time value before the predetermined threshold time th_t from the point in time at which the stressful situation 611 to 614 is identified as time attribute information of the stress context information POCC. When there is no time attribute information previously set, the processor 130 may designate the corresponding time value as initial time attribute information of the stress context information POCC.

For example, assuming a predetermined threshold time of five minutes, and a situation in which a frequency of a stressful situation of a user is recognized in a specific conference room at 3 pm, the processor 130 may designate time attribute information of “2:55 PM,” which is a time value before the threshold time, and location attribute value of “meeting room” as the stress context information POCC.

POCC=[surrounding context information={2:55 pm, conference room}]

In this case, the time attribute information “2:55 pm” included in the stress context information may be adjusted by feedback received from the user device 200 in a process of the psychological stability service being performed.

That is, the processor 130 may dynamically learn the time attribute information of the stress context information based on at least one of feedback information from the user device 200 corresponding to the psychological stability service and information about a frequency of occurrences of a stressful situation corresponding to the stress context information according to the psychological stability service.

As an embodiment, the processor 130 may, upon receiving positive feedback information from the user device 200 or identifying that the frequency of occurrence of a stressful situation decreases, maintain the time attribute information of the stress context information.

That is, subsequent to the psychological stabilization service being started, when positive feedback that may be considered helpful in inducing emotional stability is received from the user, or when it is identified that the frequency of occurrence of a stressful situation decreases after the psychological stabilization service is performed, the time attribute information in the stress context information may be maintained.

In an embodiment, the processor 130, upon receiving negative feedback information from the user device 200 or identifying that the frequency of occurrence of a stressful situation does not decrease, may allow the time attribute information in the stress context information to be learned in a direction in which positive feedback information is received from the user device 200 or the frequency of occurrences of a stressful situation decreases.

In this process, an embodiment of the present invention may allow the time attribute information of the stress context information to be learned based on various existing algorithms, such as deep learning and machine learning.

Thereafter, the processor 130 provides a preemptive psychological stabilization service to the user device 200 based on the stress context information corresponding to a stressful situation.

In this case, the stress context information may include attribute information included in the surrounding context information, such as time, place, temperature, and conversation partner information. Based on this, when attribute information of surrounding context information of multimodal user experience data collected from the user device 200 matches the stress context information, the processor 130 may provide a preemptive psychological stabilization service such that the preemptive psychological stabilization service is performed in the user device 200.

For example, when the user visits a specific place periodically and a stressful situation of the user is repeatedly recognized based on physiological signal information of the user in a situation in which the corresponding place has a specific temperature or higher, the surrounding context information regarding the “place” and “temperature” before a threshold time may be recognized as stress context information. In this case, in designating the corresponding stress context information, “time attribute information” may be specified as a specific time range according to the importance contributing to defining the stress context information or may be specified as a default value NULL to prevent a specific value from being specified. In addition, unrecognized attribute information, such as “conversation partner information” may be specified as a default value NULL.

POCC=[surrounding context information={place, temperature, . . . , }]

As another example, when a time period in which stressful situations 613 and 614 recognized based on the association between physiological signal information and behavior information overlap stressful situations 621 and 622 and 631 to 632 recognized based on speech signal information or speech text information occurs often at a frequency equal greater than or equal to a specific threshold value th_n on the multidimensional time information, the stressful situation recognized based on the surrounding context information and each experience data modality of the user before a specific threshold time from the overlap time period may be designated as stress context information POCC for a psychological stability service of the user.

As an example, during a conversation with a specific conversation partner in a specific place, when negative emotions are repeatedly recognized based on physiological signal information and speech signal information, the “place” and “conversation partner” among the surrounding context information may be specified and designated as the corresponding stress context information. Alternatively, during a conversation with the specific conversation partner in the specific place, when negative emotions frequently occur based on an utterance of the user including a specific vocabulary, the stress context information may be designated as a combination of the surrounding context information and speech text information.

POCC=[surrounding context information={place, conversation partner, . . . , }]

POCC=[surrounding context information={place, conversation partner, . . . . , }, speech text information]

As another example, speech text information 631 and 632 commonly uttered by the user at a level greater than or equal to a predetermined threshold value th_n in a stressful situation recognized based on speech signal information may be designated as stress context information POCC for the psychological stability service of the user. That is, in this case, the stress context information POCC may be information about the speech text itself frequently uttered by the user in the stressful situation. As an example, in a case of negative emotions occurring frequently when the user makes an utterance containing a specific vocabulary, such as “depression,” information 631 and 632 of the corresponding spoken sentence itself may be designated as stress context information POCC.

POCC=[speech text information]

As described above, according to the embodiment of the present invention, when multimodal user experience data collected from the user device 200 is accumulated, the association between the multimodal user experience data is analyzed to recognize a stressful situation, and a frequency pattern of the stressful situation is analyzed to recognize stress context information and, based on the stress context information, the server 100 may provide a preemptive psychological stabilization service to the user device 200.

In addition, the server 100 may, through recognition of frequent stressful situations based on pieces of multimodal user experience data which are consecutively collected, periodically update a plurality of pieces of stress context information.

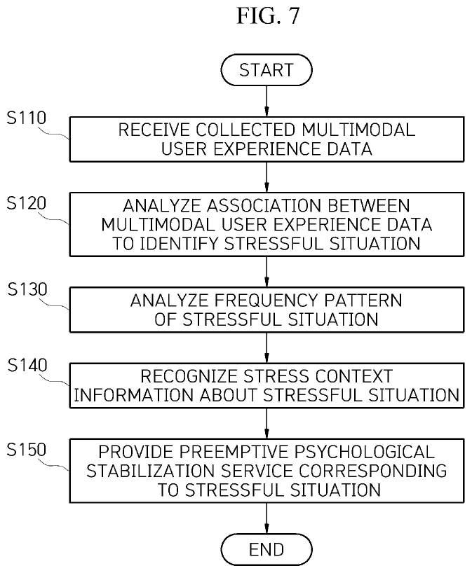

FIG. 7 is a flowchart showing a method of analyzing multimodal user experience data according to an embodiment of the present invention.

Meanwhile, each operation shown in FIG. 7 may be understood as being performed by the server 100, but the present invention is not limited thereto.

First, the server 100, upon receiving multimodal user experience data collected based on the predetermined user device 200 (S110), analyzes the association between the collected multimodal user experience data to identify whether the user is in a stressful situation (S120).

Next, the server 100, when it is identified that the user is in a stressful situation, analyzes a frequency pattern of the stressful situation of the user (S130) and, based on the frequency pattern of the stressful situation, recognizes stress context information about a stressful situation occurring to the user (S140).

Thereafter, the server 100 provides the user device 200 with a preemptive psychological stabilization service based on the stress context information corresponding to the stressful situation (S150).

Meanwhile, in the above description, operations S110 to S150 may be further segmented into a larger number of operations or combined into a smaller number of operations according to examples of implementation of the present invention. In addition, some of the operations may be omitted or may be executed in the reverse order as needed. Parts, which are omitted in the following description and have been described above with reference to FIGS. 1 to 6, may be applied to the description of FIG. 7.

The embodiment of the present invention described above may be implemented as a program (or an application) to be executed in combination with a server, which is hardware, and stored in a medium.

The program may include codes coded in a computer language, such as C, C++, Java, other machine language, etc., that may be read by a processor (a central processing unit (CPU)) of a computer through a device interface of the computer in order for the computer to read the program and execute the methods implemented as the program. The code may include a functional code that is related to a function that defines functions needed to execute the methods and may include an execution procedure related control code needed to cause the processor of the computer to execute the functions according to a predetermined procedure. In addition, the code may further include a memory reference related code as to whether additional information or media needed to cause the processor of the computer to execute the functions should be referred to at a location (an address) of an internal or external memory of the computer. In addition, when the processor of the computer needs to communicate with any other computers or servers, etc. at a remote site, to perform the above-described functions, the code may further include communication related codes such as how to communicate with any other computers or servers at a remote site and what information or media should be transmitted or received during communication.

The storage medium does not refer to a medium that stores data for a short period of time, such as a register, cache, memory, etc., but refers to a medium that stores data semi-permanently and may be read by a device. Specifically, examples of the storage medium include may include a read-only memory (ROM), a random-access memory (RAM), a compact disc (CD)-ROM, a magnetic tape, a floppy disk, an optical data storage device, etc. but are not limited thereto. That is, the program may be stored in various recording media on various servers which the computer may access or on various recording media on the computer of the user. In addition, the medium may be distributed over computer systems connected through a network so that computer-readable codes may be stored in a distributed manner.

The operations of the method or algorithm described in connection with the embodiment of the present invention may be implemented directly in hardware, implemented in a software module executed by hardware, or implemented in a combination thereof. Software modules may reside in a RAM, a ROM, an Erasable Programmable ROM (EPROM), an Electrically Erasable Programmable ROM (EEPROM), a flash memory, a hard disk, a removable disk, a CD-ROM, or any other form of computer-readable recording medium known in the art to which the present invention pertains.

As is apparent from the above, the server for providing a psychological stability service, the user device, and the method of analyzing multimodal user experience data for the same can automatically recognize and manage a point in time, at which a psychological stabilization service needs to be performed in practice, from personal experience data, and provide a personalized psychological stabilization service.

In addition, according to the present invention, the server for providing a psychological stability service, the user device, and the method of analyzing multimodal user experience data for the same can allow a psychological stability service to be provided at a preemptive point in time before a stressful situation is predicted to occur to a user so that the user can be induced to more efficient emotional stability.

The effects of the present invention are not limited to those described above, and other effects not mentioned above will be clearly understood by those skilled in the art from the above detailed description.

Although the present invention has been described in detail above with reference to the exemplary embodiments, those of ordinary skill in the technical field to which the present invention pertains should be able to understand that various modifications and alterations may be made without departing from the technical spirit or essential features of the present invention. Therefore, it should be understood that the disclosed embodiments are not limiting but illustrative in all aspects.

What is claimed is:

1. A method of analyzing multimodal user experience data for providing a psychological stability service, which is performed by a computer, the method comprising:
receiving multimodal user experience data collected on the basis of a user device that is predetermined;
analyzing an association between the collected multimodal user experience data to identify whether a user is in a stressful situation;
when the stressful situation is identified, analyzing a frequency pattern of the stressful situation of the user;
recognizing stress context information about the stressful situation occurring to the user on the basis of the frequency pattern of the stressful situation; and
providing the user device with a preemptive psychological stability service on the basis of the stress context information corresponding to the stressful situation.

2. The method of claim 1, wherein the receiving of the multimodal user experience data collected on the basis of the predetermined user device includes
receiving the multimodal user experience data including one or more of: surrounding context information of the user including information related to at least one among a time, a place, a temperature, and a conversation partner; physiological signal information of the user; behavior information of the user; speech signal information of the user; and speech text information of the user.

3. The method of claim 2, wherein the analyzing of the association between the collected multimodal user experience data to identify whether the user is in the stressful situation includes at least one of:
identifying whether the user is in a stressful situation on the basis of the surrounding context information of the user, the physiological signal information of the user, and the behavior information of the user; and
identifying whether the user is in a stressful situation on the basis of one or more of the speech signal information of the user and the speech text information of the user.

4. The method of claim 3, wherein the identifying of whether the user is in the stressful situation on the basis of the surrounding context information of the user, the physiological signal information of the user, and the behavior information of the user includes:
calculating an association index for a change of physiological signal information of the user corresponding to an activity amount of each behavior of the user; and
when the physiological signal information of the user at a specific point in time according to the surrounding context information has a variance greater than or equal to a preset threshold from the association index compared to the behavior information of the user, identifying that the user is in the stressful situation.

5. The method of claim 3, wherein the analyzing of the frequency pattern of the stressful situation of the user includes
analyzing the frequency pattern of the stressful situation including multidimensional time information including a unit time period and a unit accumulation period.

6. The method of claim 5, wherein the recognizing of the stress context information about the stressful situation occurring to the user on the basis of the frequency pattern of the stressful situation includes
recognizing, as the stress context information, at least one of the surrounding context information obtained before a predetermined threshold time from a point in time at which the stressful situation is identified and the speech text information commonly uttered by the user at a level greater than or equal to a preset threshold value.

7. The method of claim 6, wherein the recognizing of the stress context information about the stressful situation occurring to the user on the basis of the frequent pattern of the stressful situation includes
setting a time value before the predetermined threshold time as time attribute information of the stress context information.

8. The method of claim 7, wherein the time attribute information of the stress context information is dynamically learned on the basis of at least one of feedback information from the user device corresponding to the psychological stability service and information about a frequency of occurrences of a stressful situation corresponding to the stress context information according to the psychological stability service.

9. The method of claim 8, wherein, when positive feedback information is received from the user device or when the frequency of occurrences of the stressful situation decreases, the time attribute information of the stress context information is maintained.

10. The method of claim 8, wherein, when negative feedback information is received from the user device or when the frequency of occurrences of the stressful situation does not decrease, the time attribute information of the stress context information is learned in a direction in which positive feedback information is received from the user device or a direction in which the frequency of occurrences of the stressful situation decreases.

11. The method of claim 6, wherein the providing of the preemptive psychological stability service on the basis of the stress context information corresponding to the stressful situation to the user device includes,
when attribute information of the surrounding context information in the multimodal user experience data collected from the user device matches the stress context information, providing the preemptive psychological stabilization service to be performed.

12. The method of claim 6, wherein the providing of the preemptive psychological stability service on the basis of the stress context information corresponding to the stressful situation to the user device includes,
when the speech text information based on the multimodal user experience data collected from the user device is designated to the stress context information, providing the preemptive psychological stabilization service to be performed.

13. A server for providing a psychological stability service by analyzing multimodal user experience data, the server comprising:
a communication module configured to receive pieces of multimodal user experience data collected from a user device, which is predetermined, and provide the user device with a preemptive psychological stability service;
a memory in which a program for providing the user device with the preemptive psychological stability service on the basis of the multimodal user experience data is stored; and
a processor configured to execute the program stored in the memory, wherein the processor executes the program to:
analyze an association between the collected multimodal user experience data to identify whether a user is in a stressful situation;
when the stressful situation is identified, analyze a frequency pattern of the stressful situation of the user;
recognize stress context information about a stressful situation occurring to the user on the basis of the frequency pattern of the stressful situation; and
provide the user device with a preemptive psychological stability service on the basis of the stress context information corresponding to the stressful situation.

14. The server of claim 13, wherein the processor is configured to:
identify whether the user is in a stressful situation on the basis of physiological signal information of the user and behavior information of the user among the pieces of multimodal user experience data, or identify whether the user is in a stressful situation on the basis of one or more of speech signal information of the user and speech text information of the user; and
analyze the frequency pattern of the stressful situation including multidimensional time information including a unit time period and a unit accumulation period.

15. The server of claim 14, wherein the processor is configured to:
recognize, as the stress context information, at least one of surrounding context information obtained before a predetermined threshold time from a point in time at which the stressful situation is identified and speech text information commonly uttered by the user at a level greater than or equal to a preset threshold value; and
set a time value before the predetermined threshold time as time attribute information of the stress context information.

16. The server of claim 15, wherein the processor is configured to dynamically learn the time attribute information of the stress context information on the basis of at least one of feedback information from the user device corresponding to the psychological stability service and information about a frequency of occurrences of a stressful situation corresponding to the stress context information according to the psychological stability service.

17. The server of claim 16, wherein the processor is configured to, when positive feedback information is received from the user device or the frequency of occurrences of the stressful situation decreases, maintain the time attribute information of the stress context information.

18. The server of claim 16, wherein the processor is configured to, when negative feedback information is received from the user device or the frequency of occurrences of the stressful situation does not decrease, allow the time attribute information of the stress context information to be learned in a direction in which positive feedback information is received from the user device or a direction in which the frequency of occurrences of the stressful situation decreases.

19. The server of claim 15, wherein the processor is configured to provide the preemptive psychological stabilization service to be performed in at least one of:
a case in which attribute information of the surrounding context information in the multimodal user experience data collected from the user device matches the stress context information; and
a case in which the speech text information based on the multimodal user experience data collected from the user device is designated to the stress context information.

20. A user device for performing a psychological stability service on the basis of multimodal user experience data, the user device comprising:
a sensor unit configured to receive multimodal user experience data including one or more of: surrounding context information of a user including information related to at least one among a time, a place, a temperature, and a conversation partner;
physiological signal information of the user; behavior information of the user; speech signal information of the user; and speech text information of the user;
a communication module configured to transmit the multimodal user experience data to a psychological stability service providing server, and receive a preemptive psychological stability service provided from the psychological stability service providing server;
a memory in which an application for providing the preemptive psychological stability service is stored; and
a processor configured to execute the program stored in the memory to: analyze an association between the collected multimodal user experience data to identify whether the user is in a stressful situation; and when the stressful situation is identified, receive a preemptive psychological stability service based on stress context information corresponding to the stressful situation and execute the preemptive psychological stability service,
wherein the stress context information for the stressful situation occurring to the user is recognized on the basis of a frequency pattern of the stressful situation of the user.
